(19) United States
(12) Patent Application Publication
(10) Pub. No.: US 2004/0034036 A1
(43) Pub. Date: Feb. 19, 2004

(54) N-(3,5-dichloro-2-methoxyphenyl)-4-methoxy-3-piperazin-1-yl-benzenesulfonamide

(76) Inventors: Steven Mark Bromidge, Verona (IT); Stephen Frederick Moss, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/362,789

(22) PCT Filed: Aug. 27, 2001
(86) PCT No.: PCT/EP01/09927

(30) Foreign Application Priority Data
Aug. 31, 2000 (GB) 0021450.2

(51) Int. Cl. A61K 31/495

(52) U.S. Cl. 514/255.03

(57) ABSTRACT

The invention relates to a novel sulfonamide compound having pharmacological activity, a process for its preparation, to compositions containing the same and to its use in the treatment of various disorders, particularly CNS disorders.

This invention relates to a novel sulfonamide compound having pharmacological activity, a process for its preparation, to compositions containing the same and to its use in the treatment of various CNS and other disorders.

WO 98/27081 discloses a series of aryl sulfonamide compounds that are said to possess 5-HT₆ receptor activity and are useful in the treatment of various CNS disorders. WO 99/02502 describes a further class of sulfonamide derivatives which are also described as possessing 5-HT₆ receptor antagonist activity. A novel compound has now been discovered which falls within the generic scope of WO 99/02502, but is not specifically disclosed therein, and has been found to exhibit a surprisingly advantageous overall pharmacological and toxicological profile.

The present invention therefore provides, in a first aspect, N-(3,5-Dichloro-2-methoxy-phenyl)-4-methoxy-3-piperazin-1-yl-benzenesulfonamide, that is to say, the compound of formula (1):

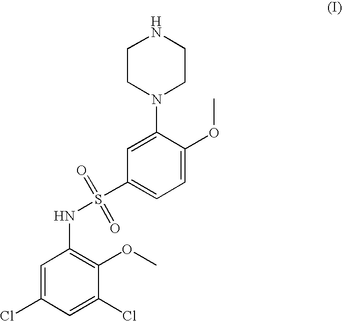

or a pharmaceutically acceptable salt thereof.

The compound of formula (I) demonstrates 5-HT₆ receptor antagonist activity. The affinity of this compound for the 5-HT₆ receptor was tested according to procedures described in WO 98/27081 and found to have a pKi of 9.1 at human cloned receptors. The selectivity of the compound of formula (I) for 5-HT₆ receptors can be determined using binding assays methods which are well known to those skilled in the art. The compound of formula (I) demonstrates a greater than 300-fold selectivity for 5-HT₆ receptors particular over other 5-HT receptor sub-types and dopaminergic receptors.

The compound of formula (I) demonstrates an advantageous pharmacological profile in that it combines high oral bioavailability along with enhanced CNS penetration relative to the structurally related compound N-(2,3,5-Trichlorophenyl)-4methoxy-3-piperazin-1-ylbenzenesulfonamide (Example 136 of WO 99/02502).

The compound of formula (I) was also evaluated in the MEST (Maximal electroshock seizure threshold) test according to procedures described by Upton et al. (British Journal of Pharmacology, 1997, 121, 1679-1686). It was found to demonstrate a strongly anti-convulsant effect. By way of contrast, the compound N-(2,5-Dibromo-3-fluorophenyl)-4-methoxy-3-piperazin-1-ylbenzenesulfonamide (Example 140 of WO 99/02502) was found to have pro-convulsant effect in this test.

The compound of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compound of formula (I) may be prepared in crystalline or non-crystalline form, and, if crystalline, may optionally be hydrated or solvated. This invention includes within its scope stoichiometric hydrates as well as a compound containing variable amounts of water.

The present invention also provides a process for the preparation of the compound of formula (I) or a pharmaceutically acceptable salt thereof, which process comprises the coupling of the compound of formula (II):

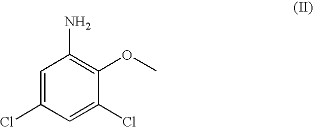

with the compound of formula (III) or a protected derivative thereof:

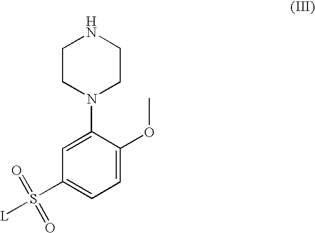

in which L is halogen and optionally thereafter:

removing any protecting groups;

forming a pharmaceutically acceptable salt.

The reaction of a compounds of formulae (II) and (III) is carried out by mixing the two reagents together, optionally in an inert solvent (such as dichloromethane, 1,2-dichloroethane, THF, acetonitrile and t-butyldimethyl ether) with or without the addition of a suitable base (such as pyridine, triethylamine or isoquinoline). Preferably the reaction of the compound of formulae (II) and (III) is carried out in dichloromethane in the presence of isoquinoline as base. Preferably L is chloro.

Those skilled in the art will appreciate that it may be necessary to protect certain groups. Suitable protecting groups and methods for their attachment and removal are conventional in the art of organic chemistry, such as those described in Greene T. W. ‘Protective groups in organic synthesis’ New York, Wiley (1981). A preferred protecting group for the piperazine group is trichloroacetyl.

The compound of formula (II) can be prepared according to procedures described by Kohn et. al. (Montash. Chem., 1928, 49,157) or by methods described herein. The compound of formula (III) or a protected derivative thereof can be prepared by methods described herein.

Pharmaceutically acceptable salts may be prepared conventionally by reaction with the appropriate acid or acid derivative.

The compound of formula (I) and its pharmaceutically acceptable salts has 5-HT₆ receptor activity and is believed to be of potential use in the treatment of certain CNS disorders such as anxiety, depression, epilepsy, obsessive compulsive disorders, migraine, cognitive memory disorders (e.g. Alzheimers disease, age related cognitive decline and mild cognitive impairment), Parkinsons Disease, ADHD (Attention Deficit Disorder/Hyperactivity Syndrome), sleep disorders (including disturbances of Circadian rhythym), feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. The compound of this invention is also expected to be of use in the treatment of certain GI (gastrointestinal) disorders such as IBS (irritable Bowel Syndrome).

Thus the invention also provides, in a further aspect, the compound of formula (I) or a pharmaceutically acceptable salt thereof, for use as a therapeutic substance, in particular in the treatment or prophylaxis of the above disorders. More particularly, the invention provides for the compound of formula (I) or a pharmaceutically acceptable salt thereof, for use in the treatment of Alzheimers disease, age related cognitive decline, ADHD, depression and/or anxiety.

The invention further provides a method of treatment or prophylaxis of the above disorders, in mammals including humans, which comprises administering to the sufferer a safe and therapeutically effective amount of the compound of formula (I) or a pharmaceutically acceptable salt thereof.

In another aspect, the invention provides the use of the compound of formula (I) or a pharmaceutically acceptable salt thereof in the manufacture of a medicament for use in the treatment or prophylaxis of the above disorders, particularly CNS disorders.

In order to use the compound of formula (I) in therapy, they will normally be formulated into a pharmaceutical composition in accordance with standard pharmaceutical practice. The present invention also provides a pharmaceutical composition, which comprises the compound of formula (I) or a pharmaceutically acceptable salt thereof, and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three times a day, so that the total daily dosage is in the range of about 0.5 to 100 mg; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following descriptions and examples illustrate the preparation of the compound of the invention.

### DESCRIPTION 1

### 1-(2-Methoxyphenyl)-4-trichloroacetylpiperazine (D1)

A solution of 1-(2-methoxyphenyl)piperazine (7.0 g) in dichloromethane (30 ml) was added over 0.25 h to a stirred solution of trichloroacetyl chloride (4.06 ml) in dichloromethane (40 ml) at room temperature under argon. Diisopropylethylamine (5.95 ml) was then added and the whole was stirred for 18 h. The reaction mixture was washed with water (2×100 ml), dried (Na₂SO₄) and concentrated to give the title compound (D1) as an oil (11.2 g, 91%), MS: m/z (MH⁺) 337/339.

### DESCRIPTION 2

### 3-(4-Trichloroacetylpiperazin-1-yl)-4-methoxybenzenesulfonyl Chloride (D2)

A solution of 1-(2-methoxyphenyl)-4-trichloroacetylpiperazine (D1) (10 g) in dichloromethane (115 ml) was added over 0.3 h to ice-cooled chlorosulfonic acid (52 ml). After 0.5 h at 0° C. then 1 hour at room temperature, the solution was poured onto a mixture of ice-water (500 g) and dichloromethane (500 ml) with rapid stirring. The layers were separated and the organic phase was washed with water (2×800 ml), dried (MgSO₄) and concentrated to give the title compound (D2) as a foam (6 g, 46%), MS: m/z (MH⁺) 435/437.

### DESCRIPTION 3

### 1,5-Dichloro-2-methoxy-3-nitro Benzene (D3)

A stirred suspension of potassium carbonate (99.7 g), iodomethane (89 ml) and 2,4-dichloro-6-nitrophenol (containing 20% water)(100 g) in N,N-dimethylformamide (1 L) was heated at 60° C. for 18 h. The reaction mixture was cooled and the solid was filtered-off and washed with dichloromethane (2×500 ml). The filtrate was evaporated in vacuo to an oily solid which was taken-up in dichloromethane (1.5 L). The combined organics were washed with 1M sodium hydroxide (1.5 L), then water (1 L). The organic phase was dried (MgSO₄) and concentrated to give the title compound (D3) as a solid (35.7 g, 42%). MS: m/z (M-H⁻) 221/223.

### DESCRIPTION 4

### 3,5-Dichloro-2-methoxy-phenylamine (D4)

A vigorously stirred suspension of iron powder (42.5 g), 1,5-dichloro-2-methoxy-3-nitro benzene (D3) (65 g) in methanol (500 ml) and saturated aqueous ammonium chloride solution (700 ml) was heated at reflux for 3 h. The mixture was filtered and the solid washed with dichloromethane/methanol (1:1) (4×150 ml) then dichloromethane (4×200 ml). The filtrate was diluted with water (500 ml), shaken and the layers separated. The aqueous layer was further extracted with dichloromethane (500 ml) and the combined organic extracts were dried (Mg SO₄) and concentrated to an oil. The oil was purified by column chromatography on silica eluting with dichloromethane/hexane (4:1) then dichloromethane to afford the title compound (D4) as an oil (41.8 g, 74%). MS: m/z (M⁺) 191/192.

### DESCRIPTION 5

### N-(3,5-Dichloro-2-methoxy-phenyl)-4methoxy-3-[4(2,2,2-trichloro-ethanoyl)-piperazin-1-yl]-benzenesulfonamide (D5)

A solution of 3,5-dichloro-2-methoxy-phenylamine (D4) (41 g), 3-(4-trichloroacetylpiperazin-1-yl)-4-methoxybenzenesulfonyl chloride (D2)(93 g) and dry pyridine (51.7 ml) in dry 1,2-dichloroethane (1.5 L) was refluxed for 40 h under argon. The reaction mixture was cooled to room temperature and washed with 1 M hydrochloric acid (1.5 L), water (2×1.5 L), dried (MgSO₄) and concentrated in vacuo to an oil. The oil was stirred with hot ethanol (400 ml) to give the title compound (D5) as a cream solid which was filtered and washed with cold ethanol then diethyl ether (104.8 g, 83%).

¹H (400 MHz, CDCl₃) δ1.84-1.87 (2H, m), 3.08-3.10 (4H,m), 3.64 (3H, s), 3.73-376 (2H, m), 3.93 (3H, s), 6.91 (1H, d, J 8.4 Hz), 7.04 (1H, d, J 2.4 Hz), 7.14 (1H, s), 7.30 (1H, d, J 2.4 Hz), 7.53-7.57 (2H, m); MS: m/z (MH⁺) 590/592/594.

The compound D2 can also be obtained by the following procedure:

### DESCRIPTION 2a

### 3-(4-Trichloroacetylpiperazin-1-yl)-4-methoxybenzenesulfonyl Chloride (D2a)—Alternative Procedure to D1/D2

1-(2-methoxyphenyl)piperazine hydrochloride was treated with trichloroacetyl chloride (2.04 eq), added portionwise, in dichloromethane solution in the presence of diisopropylethylamine (1.02 eq). The mixture was stirred at 20 to 22° C. for 30 minutes at which point the reaction was shown to be complete by HPLC analysis. The resulting reaction mixture was washed with water, then the aqueous phase back extracted with dichloromethane. The combined organic phases were washed with water then dried over sodium sulfate and filtered through Celite (Diatomaceous Earth). The filtrate was added to chlorosulfonic acid over 100 minutes at −9 to 13° C. then stirred at 13 to 21° C. for 17.5 hours. The resulting solution was then added to a pre-cooled (1° C.) mixture of dichloromethane and process water over ca. 2.5 hours at 0 to 18° C. The phases were separated and the aqueous phase was extracted with dichloromethane then the combined organic phases washed with water. After clarification through an in-line filter the organic solution was heated to reflux and dichloromethane exchanged for toluene by put-and-take distillation. The toluene solution was then cooled to 18° C. and diluted with in-heptane to precipitate the product, which was collected by centrifugation and dried to give the title compound.

The compound D4 can also be obtained by the following procedure.

### DESCRIPTION 4a

### 3,5-Dichloro-2-methoxy-phenylamine (D4a)—Alternative Procedure to D3/D4

2,4-dichloro-6-nitrophenol was dissolved in DMF and treated with dimethylsulfate (3.3 eq), added over 55 minutes, in the presence of potassium carbonate (˜2.8 eq), then stirred at 35-40° C. for 3 hours. The mixture was cooled to 25° C. then partitioned between n-heptane and aqueous ammonia. The lower aqueous layer was re-extracted with n-heptane then the two organic layers were combined and washed sequentially with 10% aqueous potassium carbonate solution and water. The organic solution was then hydrogenated over 1% platinum on carbon, type 156, 50% paste at 15-25° C. and 40-47 psig hydrogen until the reaction was shown to be complete by HPLC. The mixture was filtered through Celite (Diatomaceous Earth) then evaporated to dryness under reduced pressure at to give the title compound as an oil.

The compound D5 can also be obtained by the following procedure.

### DESCRIPTION 5a

### N-(3,5-Dichloro-2-methoxy-phenyl)-4-methoxy-3-[4-(2,2,2-trichloro-ethanoyl)-piperazin-1-yl]-benzenesulfonamide (D5a)—Alternative Procedure to D5

3-(4-Trichloroacetylpiperazin-1-yl)-4-methoxybenzenesulfonyl chloride (D2)(1.0 equiv) was suspended in dichloromethane (0.9 vols) with stirring and 3,5-Dichloro-2-methoxy-phenylamine (D4) (1.05 equiv) added. A solution of isoquinoline (1.5 equiv) in dichloromethane (0.2 vols) was added in four portions maintaining the temperature between 17 and 26° C. The mixture was heated to reflux for 2 hours 15 minutes. The solvent was exchanged for ethanol (3.9 vols) by put and take distillation. The suspension was cooled to 0 to 5° C. and stirred for 1 hour. The title product was isolated by filtration, washed with ethanol (1.5 vols) and dried at 30 to 35° C. under vacuum

### EXAMPLE 1

### N-(3,5-Dichloro-2-methoxy-phenyl)-4-methoxy-3-piperazin-1-yl-benzenesulfonamide (E1)

A 1M solution of potassium hydroxide (609 ml) was added over 5 minutes to a rapidly stirred solution of N-(3,5-dichloro-2-methoxy-phenyl)-4-methoxy-3-[4-(2,2,2-trichloro-ethanoyl)-piperazin-1-yl]-benzenesulfonamide (D5)(103 g) in tetrahydrofuran (1.5 L) at room temperature. After stirring for 18 h, the stirred, ice-cooled mixture was adjusted to pH 7.0 by the addition of concentrated hydrochloric acid to afford the title compound (El) as a cream solid which was filtered, washed with water (3 x 100ml) and dried (72.9 g, 94%).

¹H (400 MHz, DMSO-D₆/CD₃OD4:1) δ2.95-3.15 (8H, m), 3.63 (3H, s), 3.82 (3H, s), 6.88 (1H, br d), 7.0 (1H, br dd), 7.18 (1H, br d), 7.29 (1H, br d), 7.40 (1H, br dd); MS: m/z (MH⁺) 446/448. m.p. 189-90° C.

### EXAMPLE 2

### N-(3,5-Dichloro-2-methoxy-phenyl)-4-methoxy-3-piperazin-1-yl-benzenesulfonamide Hydrochloride (E2)

N-(3,5-Dichloro-2-methoxy-phenyl)-4-methoxy-3-piperazin-1-yl-benzenesulfonamide (E1) (20 g) was suspended in ethanol (200 ml) at room temperature and to the stirred suspension was added conc. hydrochloric acid (density=1.18, 4.3 ml, 1.1 equivalents) over 1 minute. The resulting solution was left to stand for 24 h at 0° C. to give the title compound (E2) as a white solid (16.4 g, 76%).

¹H (400 MHz, DMSO-d₆) δ3.18 (8H, br s), 3.53 (3H, s), 3.86 (3H, s), 7.12 (1H, d, J 8.4 Hz), 7.32 (1H, d, J 2.4 Hz), 7.36 (1H, d, J 2.4 Hz), 7.40 (1H, d, J 2.4 Hz), 7.46 (1H, dd, J 2.4, 8.4 Hz), 9.4 (2H, br s), 10.0 (1H, br s); MS: m/z (MH⁺) 446/448. m.p. 207-9° C.

### EXAMPLE 3

### N-(3,5-Dichloro-2-methoxyphenyl)-4-methoxy-3-piperazin-1-yl-benzenesulfonamide 4-Toluenesulfonate (E3)

A solution of 4-toluenesulfonic acid monohydrate (10.7 g, 56 mmole) in ethanol (75 ml) was added to a stirred suspension of N-(3,5-dichloro-2-methoxyphenyl)-4-methoxy-3-piperazin-1-yl-benzenesulfonamide (E1) (25 g, 56 mmole) in ethanol (400 ml) at reflux. The resulting clear pale yellow solution was then allowed to cool with stirring. The solid product was collected by filtration and dried at ambient temperature and reduced pressure to constant weight to give the title compound (E3) as a white crystalline solid (28 g, 81%).

¹H NMR (400 MHz, DMSO-d₆): δ2.29 (3H, s), 3.13 (4H, br s), 3.36 (4H, br s), 3.53 (3H, s), 3.86 (3H, s), 7.12 (3H, m), 7.33 (1H, d, J 2.4 Hz), 7.37 (2H, m), 7.48 (3H, m), 8.68 (1H, br s), 10.12 (1H, s).m.p. 207-209° C.

1. N-(3,5-Dichloro-2-methoxy-phenyl)-4-methoxy-3-piperazin-1-yl-benzenesulfonamide, that is to say, the compound of formula (I): 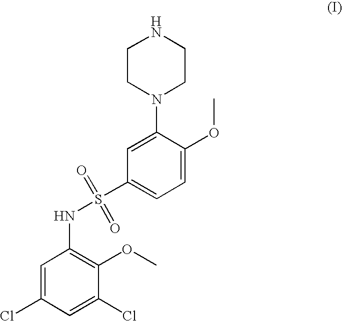
or a pharmaceutically acceptable salt thereof.

2. N-(3,5-Dichloro-2-methoxy-phenyl)-4-methoxy-3-piperazin-1-yl-benzenesulfonamide hydrochloride.

3. N-(3,5-Dichloro-2-methoxy-phenyl)-4-methoxy-3-piperazin-1-yl-benzenesulfonamide 4-toluenesulfonate.

4. A process for the preparation of the compound of formula (I) or a pharmaceutically acceptable salt thereof, which process comprises the coupling of the compound of formula (II): 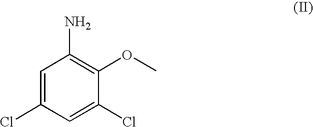
with a compound of formula (III) or a protected derivative thereof; 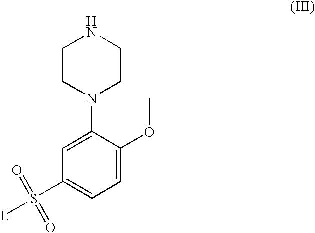
in which L is halogen and optionally thereafter:
removing any protecting groups;
forming a pharmaceutically acceptable salt.

5. N-(3,5-Dichloro-2-methoxy-phenyl)-4-methoxy-3-piperazin-1-yl-benzenesulfonamide or a pharmaceutically acceptable salt thereof for use in therapy.

6. N-(3,5-Dichloro-2-methoxy-phenyl)-4-methoxy-3-piperazin-1-yl-benzenesulfonamide or a pharmaceutically acceptable salt thereof for use in the treatment of Alzheimers disease, age related cognitive decline, ADHD, depression and/or anxiety.

7. A pharmaceutical composition which comprises N-(3,5-Dichloro-2-methoxy-phenyl)4methoxy-3-piperazin-1-yl-benzenesulfonamide or a pharmaceutically acceptable salt thereof and a pharmaceutically acceptable carrier or excipient.

8. A pharmaceutical composition which comprises N-(3,5-Dichloro-2-methoxy-phenyl)4-methoxy-3-piperazin-1-yl-benzenesulfonamide 4-toluenesulfonate and a pharmaceutically acceptable carrier or excipient.

9 The use of N-(3,5-Dichloro-2-methoxyphenyl)methoxy-3-piperazin-1-yl-benzenesulfonamide or a pharmaceutically acceptable salt thereof in the manufacture of a medicament for use in the treatment of CNS disorders.

10. A method of treatment or prophylaxis of CNS disorders in mammals which comprises administering to the sufferer a safe and therapeutically effective amount of N-(3,5-Dichloro-2-methoxy-phenyl)-4-methoxy-3-piperazin-1-yl-benzenesulfonamide or a pharmaceutically acceptable salt thereof.
